(19) United States
(12) Patent Application Publication
(10) Pub. No.: US 2025/0235683 A1
(43) Pub. Date: Jul. 24, 2025

(54) SUPERFICIAL TEMPORAL ARTERY RETROGRADE ACCESS

(71) Applicant: ALTERNATE ACCESS, Seattle, WA (US)

(72) Inventor: Akshal Patel, Seattle, WA (US)

(21) Appl. No.: 19/024,067

(22) Filed: Jan. 16, 2025

Related U.S. Application Data
(60) Provisional application No. 63/622,693, filed on Jan. 19, 2024.

(51) Int. Cl. A61M 39/02

(52) U.S. Cl. CPC A61M 39/0247; A61M 39/0208; A61M 2039/0226; A61M 2039/0258; A61M 2039/0273

(57) ABSTRACT

Superficial Temporal Artery Retrograde (STAR) access processes, systems and devices to deliver therapies retrograde, namely (up the body) via the Branches of the External and internal carotid arteries; or antegrade (down the body) to Face; Heart; Organs and extremities of the peripheral vasculature.

### PRIORITY CLAIM AND FIELD OF THE INVENTION

This case claims the priority benefit of U.S. Provisional Application No. 63/622,693 filed Jan. 19, 2024, in favor of the instant assignee with full preservation of rights under the Paris Convention, inter alia.

The field of the invention is compositions, devices, and methods of use and manufacturing of stents and other thrombectomy-type devices, in terms of structure (specifically focused upon their function) especially as it relates to endovascular or surgical management of thrombi within subject blood vessels. Those skilled in the art understand this definitional approach to likewise embrace any related art used during procedures having for example antegrade blood flow, wherein consequences of previously engaged emboli becoming partially or fully disengaged are dire.

### OBJECTS AND SUMMARY OF THE INVENTIONS

The present inventions comprise novel delivery systems for treatment of head and neck pathology.

As heretofore undisclosed or developed clinically SUPERFICIAL TEMPORAL ARTERY RETROGRADE (STAR) Access provides plethoric advantages over existing standards of care, particularly in light of no arch, great vessel crossing; creation of an immediate portal to any head and neck anatomy; no need for heparization making the procedure out-patient and eliminating fulsome access complications. MMA embolization for SDH treatment is an example of how the instant flow dynamics linked to the solutions claimed herein and embraced within the scope of the instant application for United Staters Letters Patent as herewith first disclosed in any form or reduced to practice by this written description.

### BRIEF DESCRIPTION OF THE DRAWINGS

Various preferred embodiments are described herein with references to the drawings in which merely illustrative views are offered for consideration, whereby:

FIG. 1 shows a schematic of a highly occluded vessel, illustrating lesions in the target vessel and how they are attached to anatomy accessed by the STAR access technique explained herein;

FIG. 2 likewise show what Artisans understand to be a completion angiogram in schematic of said previously highly occluded vessel, illustrating therapy applied to and ameliorative of said lesions in the target vessel and mindful of how they were shown in FIG. 1, as then attached to anatomy accessed by the STAR access technique explained herein;

FIG. 3 denoues the entire flow path of an embodiment of the STAR access approach to delivery therapy;

FIG. 4 depicts with flow charts and overlay/Venn-prediagrams aspects of the teachings of the present inventions accessed by the STAR access technique taught herein;

FIG. 5 depicts with flow charts and overlay/Venn-prediagrams aspects of the teachings of the present inventions accessed by the STAR access technique taught herein;

FIG. 6 depicts with flow charts and overlay/Venn-prediagrams aspects of the teachings of the present inventions accessed by the STAR access technique taught herein.

Artisans understand the relational bases of the scope based upon the teachings herein, it is respectfully proposed. Resorbable and eluting means along with protein capture and deposition (for example sufloxybetaines et seq.) and all image enhancement and visualization are likewise contemplated as within the scope of the therapies proposed herein and claimed below.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION OF THE INVENTIONS

The present inventors have discovered that novel and enhanced blood flow effects are instantiated in and achieved with the instant process and related devices to effectuate safe and effective delivery therapy using retrograde blood flow particularly by Superficial Temporal Access.

The description includes information that may be useful in understanding the present invention. It is not an admission that any of the information provided herein is prior art or relevant to the presently claimed invention, or that any publication specifically or implicitly referenced is prior art.

As claimed below, MMA treatments and therapy solutions to plethoric disease states are de-limited according to the instant teachings.

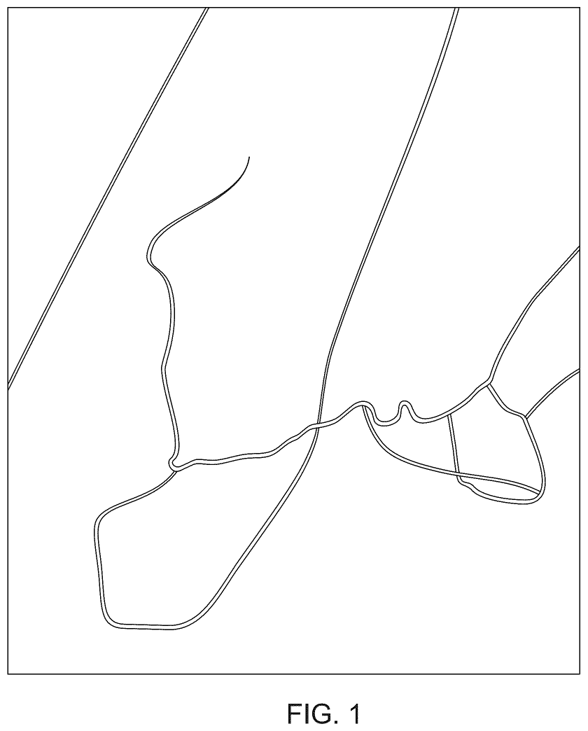

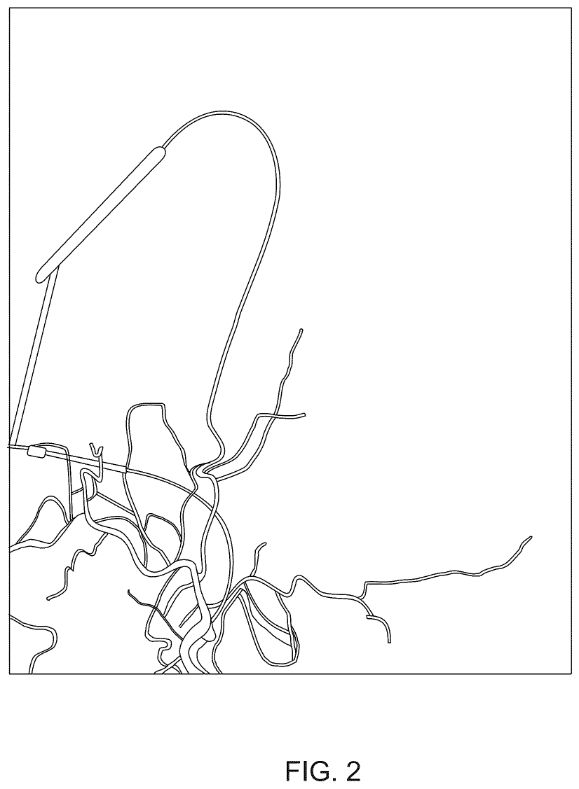

Now referring to FIG. 1 and to FIG. 2, it is shown that by using STAR access, therapy may be delivered to a brain vessel in crisis (See FIG. 1), which is then addressed and ameliorated enough to sustain adequate blood flow as shown in the schematic based on a completion angiogram (See FIG. 2).

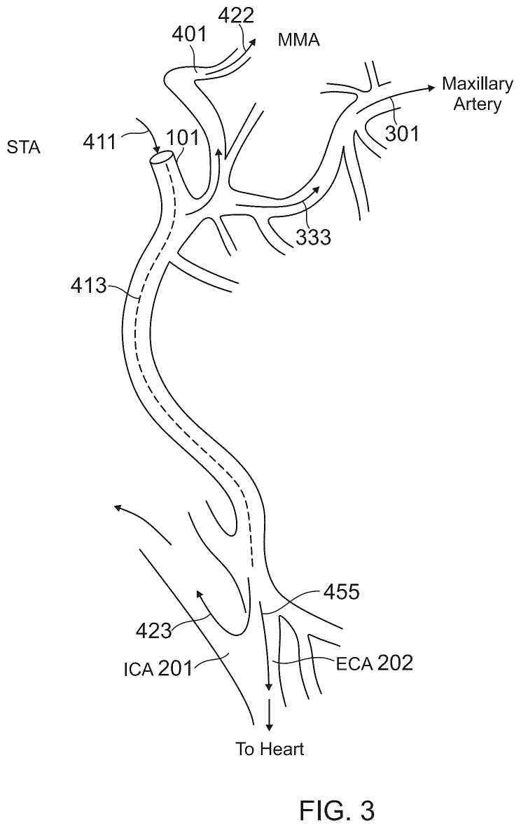

FIG. 3 shows a preferred usage of procedures and therapied by providing at least a STAR kit, Ultrasound imaging and updating in real time & from studies, registered and data compliant, access needle and wire and Sheath emplacement; diagnostic and treatment catheter-based therapy deliveries; and

Optional closure device use to complete trans-STA access procedure via the External Carotid System as the direct access platform both Intracranial and Extra cranial routes, and further comprised of specialty catheter devices which may be used and therapies delivered therein, namely (up the body) via the Branches of the External and internal carotid arteries & (down the body) to Face; Heart; Organs and extremities of the peripheral vasculature.

As shown by reference designators wherein arrow 411 shows the direction of travel for the systems of the present invention retrograde (arrow to the dotted line 413) in the direction of heart 455 and coronary vessels. Internal carotid artery (ICA) 422, and arrow 423 showing this direction of travel, reveals that ICA 422, and ECA 401 comprises the untapped highway of the circulation Artisans understand that this shows the middle meningeal artery (MMA) 401, and direction of travel 422, along with maxillary artery 301 and direction of travel 333 to the maxillary system further comprising nose, face, and eyes (not shown) 333.

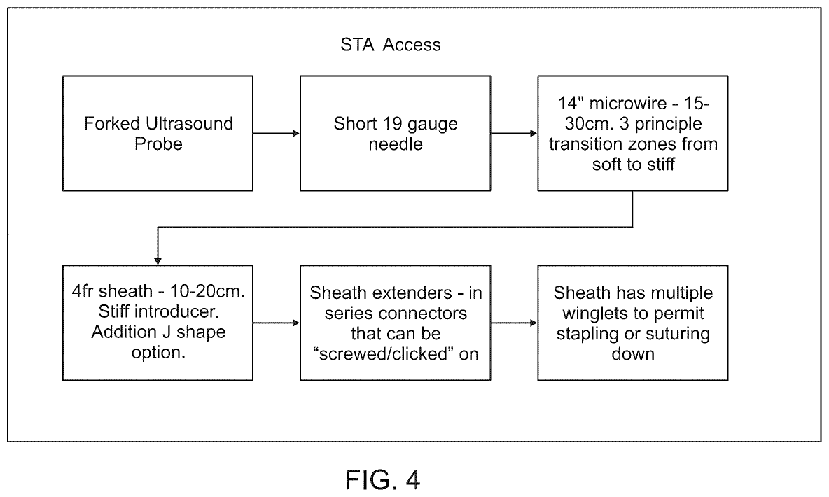

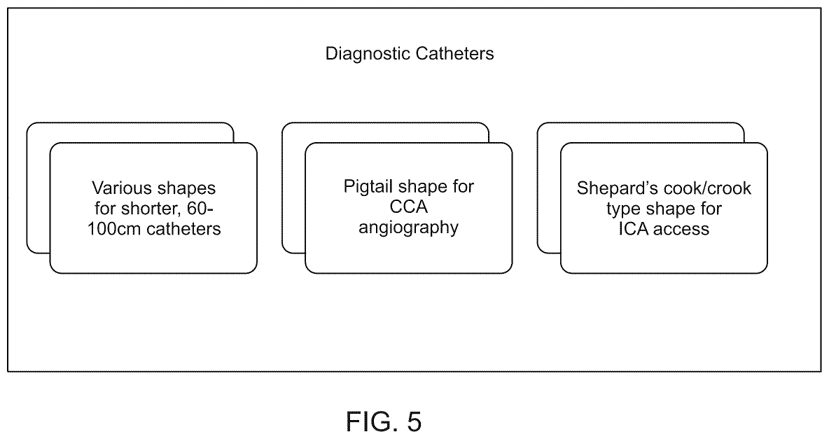

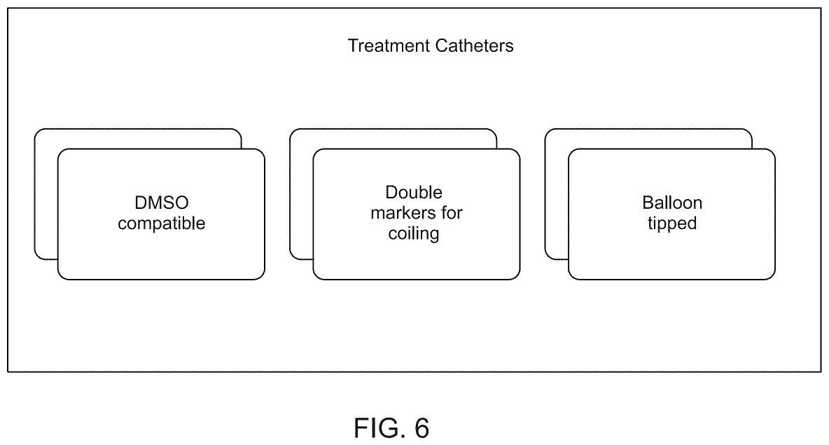

Both systems and processes enable the multiple directions summarized well by FIG. 3, with the equipment specified in FIGS. 4, 5 and 6 namely, internal carotid artery (ICA) 20, external carotid artery (ECA) 202, perhaps offers the largest number of untapped procedural iterations.

Those skilled in the art understand medications can then be eluted into the circulation directed and positioned to travel into the internal carotid artery (ICA) 201. Likewise, in addition to their primary role supplying the viscera and the head and neck and the dura, branches of the external carotid artery (ECA) 202 are important sources of collateral blood flow in cases of external carotid artery ECA), internal carotid artery (ICA), and vertebral artery stenosis or occlusion. Said collateral pathways take various forms (not shown) from internal carotid artery (ICA) to external carotid artery (ECA, from external carotid artery (ECA) to internal carotid artery (ICA, and external carotid artery (ECA) to vertebral artery. Flow may pass in either direction depending on the pattern of vascular stenosis or occlusion. FIG. 3 likewise shows how the superficial temporal artery (STA) 101 supplies the scalp, branches of the internal maxillary artery 301, and supply the cranial dural matter and middle meningeal artery (MMA), extracranial musculature (deep temporal arteries) sino-nasal region and maxillary alveolus, and palate and portions of the face. The same as occipital artery (OA) services the entire rear side of the cranial coruscation, working lockstep with the STA access.

Referring now to FIGS. 4-6 the process and kits of claim 1, further comprising, in combination:
Micropuncture sets having at least about 30-40 CM 0.014′ (inch) microwires;
SEr sheaths having shapes according to the disclosure of US 2022/0134061;
Bident-types of forked devices to stabilize the system by holding a subject vessel in place during access; Balloon catheters; infusion catheters & liquid embolic, stent & coiling options in terms of therapy devices;
Sensors and ultrasound probes, alternately being Bident-types of forking devices as above and below, and otherwise configured per the application of the processes in play.

It is respectfully pro-posed and further submitted that each of the FIGS. 4/5/6 further highlight and underscore these issues.

STAR access, it is respectfully proposed comprises progress in science and their useful arts because it uses retrograde flow to access the brain and the face and the alternatives for other vessel system from the heart to the periphery.

It is axiomatic that the instant teachings overcome the longstanding problem that during thrombectomy with antegrade blood flow, engaged emboli can become dislodged from the mechanical device. Blood flow has a water-hammer effect on the emboli which can break pieces of the emboli off or entirely dislodge the emboli from the capturing device. Avoiding this until now dangerous progression is among the objects of the instant teachings, it is respectfully submitted. In most cases, the dislodged emboli or fragments thereof can either re-occlude the original vessel or occlude a new vessel or territory.

By way of further exemplary embodiments, the present inventive subject matter is drawn to compositions, devices, and methods of making the specialty catheter products of the instant inventions.

Various objects, features, aspects and advantages of the inventive subject matter will become more apparent from the following detailed description of preferred embodiments, along with the accompanying drawing figure.

The inventor has also discovered that thrombectomy devices with improved functional characteristics, and especially with improved thrombus retention can be prepared by adjusting the diameters of the device.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A process for Superficial Temporal Artery Retrograde (STAR) access, being comprised of, in combination:
providing at least a STAR kit;
conducting Ultrasound imaging and updating in real time & from studies, registered and data compliant;
facilitating access needle and wire and sheath emplacement;
inserting diagnostic and treatment catheter-based therapy deliveries; and
deploying an optional closure device use to complete trans-STA access procedure via the External Carotid System as the direct access platform both Intracranial and Extra cranial routes.

2. The process of claim 1, further comprised of at least one specialty catheter device selected from the group of diagnostic and treatment catheters to deliver therapies retrograde, namely (up the body) via the Branches of the External and internal carotid arteries.

3. The process of claim 2, the diagnostic catheters being at least one of shorter systems between at least about 50 and 110 centimeters in length; pigtail shape for CCA angiography; and shephard's cook/crook type of shape for ICA access.

4. The process of claim 2, the treatment catheters being at least one of DMSO compatible; double markers for coiling; and balloon tipped.

5. The process of claim 1, the STAR kit being further comprised of:
at least a forked ultrasound probe;
a short needle between 18 and 21 gauge;
compatible microwires;
at least a 4 french sheath;
a stiff introducer; and
sheath extenders.

6. The process of claim 5, used to address MMA embolization for subdural hematoma treatment.

7. The process of claim 5, used to address treatment of nose bleeds.

8. The process of claim 5, used to address embolization of intracranial tumor(s).

9. The process of claim 5, used to address treatment of dural arteriovenous fistula(s).

10. The process of claim 5, used to address treatment of lingual tumor(s).

11. The process of claim 5, used to address carotid body tumor embolization.

12. The process of claim 5, used to address MMA-based headaches.

13. The process of claim 1, further comprised of at least one specialty catheter device selected from the group of diagnostic and treatment catheters to deliver therapies antegrade (down the body) to Face; Heart; Organs and extremities of the peripheral vasculature.

14. A STAR access and treatment kit, comprised of, in combination:
specialty catheters being tubular members having “J” and “U” shaped tubular members.

15. The STAR access and treatment kit, of claim 14, further comprising said specialty catheters being able to navigate into ECA branches and the Cartotid bulb.

16. The STAR access and treatment kit, of claim 15, comprised of, in combination:
a specialized ultrasound probe being fork shaped (biramous and diverged at a distal end).

17. The STAR access and treatment kit, of claim 16, further comprised of, in combination:
Micropuncture sets having at least about 30-40 CM 0.014′ (inch) microwires.

18. The STAR access and treatment kit, of claim 17, further comprised of, in combination:
4 or 5 french sheaths, with introducers having a prescribed stiffness measure by respective Young's moduli.

19. The STAR access and treatment kit, of claim 18, further comprised of, in combination:
treatment types of treatment catheters being balloon catheters; infusion catheters; liquid embolic delivery mechanisms and devices for delivering stents.

20. The STAR access and treatment kit, of claim 18, further comprised of, in combination:
Embolic coil delivery systems.
